(19) United States
(12) Patent Application Publication
(10) Pub. No.: US 2007/0016451 A1
(43) Pub. Date: Jan. 18, 2007

(54) Method for early recognition of complications associated with diagnosed medical problems

(76) Inventor: James Lee Tilson, Detroit, MI (US)

Correspondence Address: HARNESS, DICKEY & PIERCE, P.L.C., P.O. BOX 828, BLOOMFIELD HILLS, MI, 48303, US

(21) Appl. No.: 11/183,080

(22) Filed: Jul. 15, 2005

(51) Int. Cl. G06F 19/00; A61B 5/00

(52) U.S. Cl. 705/3; 600/300

(57) ABSTRACT

A method and chart for alerting a medical caregiver to undetected or developing complications associated with, or resulting from, initially diagnosed or suspected medical problems. First, a list is prepared of possible complications that might occur based upon the initial diagnosis. Signs of the complications are concisely listed on the chart. The concise chart is posted at, or is otherwise available or near the patient's location, such as at a hospital bed or nurse's station, or other easily observable locations. Thereafter, various caregiver personnel who attend to, or routinely visit, the patient for performing various services, routinely check the listed signs against the patient's condition when the patient is visited. Each caregiver person records the presence of, and any observed changes in, the listed signs that are observed giving the time of the observation. Lastly, an appropriate caregiver physician is alerted to the noted signs for diagnosing and prescribing appropriate treatments. These complications which are present or are developing in the patient can be recognized quickly and appropriate treatment may be undertaken immediately when needed.

### BACKGROUND OF THE INVENTION

This invention relates to an improved method for alerting medical caregivers, such as a patient's treating physician, to earlier recognize and treat initially undetected medical complications, or an initially undetected worsening of the patient's condition, suffered by their hospitalized patients, so as to minimize injuries resulting from complications.

In general, the conventional method for determining the presence of medical complications or medical problems suffered by a patient involves consideration, by a suitably trained medical caretaker, such as the patient's attending or treating physician, of the patient's medical history, observable physical conditions, the results obtained from relevant laboratory tests, x-rays and information obtained from various kinds of medical test equipment, and the caretaker's experience. Upon such consideration, an attending physician or other appropriately trained medical caregivers ordinarily determines or diagnoses the particular medical problems suffered by their patients and propose such treatments that are proper in their judgments.

However, there may be delays in recognizing complications or worsening of a patient's condition because often only the attending physician considers and exercises a required medical judgment. Typically the only time anyone attempts to exercise such a medical judgment is during the physician's “Rounds” that commonly occur only once, or twice, a day. Thus, there may be a substantial time gap between the onset of observable signs of a complication and the observation of, and the recognition of, the significance of such signs.

Moreover, the signs or symptoms of a complication or a developing complication resulting from a particular medical problem may be distributed among different caretaker personnel or distributed throughout voluminous medical records, thus making early recognition of potentially dangerous combinations of symptoms more difficult. Nurses who care for a large number of patients with a wide variety of medical problems and different attending physicians may not become aware of warning signs or the significance of a particular sign which they might see. Also, some nurses and providers other than the attending physicians may lack the expertise or the time to understand and to look for relevant signs. Patients may receive care from a number of different providers including therapists, nurses, nurses' aides, technicians, interns, consultants, residents, etc. who normally would not be aware of, or would not look for signs.

Hospitals and other medical care facilities usually make and keep records of a patient's history, results of test procedures, diagnoses and symptoms and prior treatments. These records are used in considering a patient's condition and in diagnosing a patient's medical problems and prescribing treatments that are provided to the patient. An example of such records, which is designed to provide relatively complete, detailed, and useful information for diagnostic purposes, is disclosed in U.S. Patent Application Publication No. US2002/0077865 A1 of Jun. 20, 2002. This disclosure describes a lengthy, detailed chart-like record system which contains a large volume of detailed medical information. This information is made available to, and is helpful to, an attending physician in diagnosing and treating a patient's problem. However, because it is so detailed and complete and lengthy, it is not practical for use by busy nurses or other hospital attendants to look for, or to be fully aware of, and to recognize quickly, significant signs of, possible complications. Thus, they are frequently unable to give a prompt alarm concerning possible undetected or developing complications that may be associated with a particular patient's diagnosed or suspected medical problems.

At times, there may be complications, that is, other medical problems which are associated with a particular diagnosed or suspected medical problem, or which may later develop following an initial diagnosis, which were not initially detected. Thus, observation and recognition of such complications normally must await first, the appearance of relevant signs or symptoms and later, a re-examination and consideration by an attending physician to diagnose the complication. Once signs or symptoms of complications are observed, recognized and properly considered, adjustments or additions to the initial diagnosis can be made and treatments of such additional complications can be undertaken. Hence, the sooner the signs are recognized and called to the attention of an appropriate attending physician, the better.

The detection and recognition of the possible presence of initially unperceived or latent or undeveloped complications may take considerable time following an initial diagnosis of a particular medical malady. Moreover, the individual signs or symptoms of such complications, even when initially observed, may not be enough for hospital personnel to recognize the presence of a complication. At times, a collection of important or significant signs, considered together, are needed to cause recognition of their significance in warning of a present or developing complication that is associated with, or results from, the initially diagnosed problem. Accordingly, there can be considerable delays in detecting and treating complications. That also delays timely treatment.

Sometimes delays in treating complications may result in severe or irreversible injury to a particular patient. In some instances, such injuries may be avoided or minimized if a particular complication is more quickly diagnosed. Thus, there is a need for a method for more rapidly recognizing, injury-causing, latent or developing complications that are associated with or result from an initially diagnosed malady. Preferably, the recognition method should be usable by other hospital personnel who are attending or visiting a patient, and who can give an alarm to the attending physician.

At times, there are observable signs or symptoms of an oncoming complication that might be observable to a casual visitor to a patient, such as a nurse's aide or nurse bringing medicines or food to a patient or adjusting the bed or other hospital attendants or visitors, etc. But, individual or separately observed signs noticed by different people, might be explainable or not understood by the observer. Moreover, observations of such a sign by a visiting attendant or other caretaker personnel or casual visitor might not be noticed by them since they would normally not be looking for such signs. Moreover, even observed signs might not be significant to them. A number of or combination of recognized signs, when observed together might be helpful in making the observer aware of their significance. But, to someone who is not medically trained, even a combination might be ignored because the observer does not recognize the meaning or significance. Thus, observed individual signs or combinations of signs might not be taken seriously. More importantly, those warning signs of a complication may not be brought to the early attention of the attending physician who is trained to recognize their significance. And at times, combinations of relevant signs, including changes in signs, are needed to alert or warn of an oncoming complication and to enable even a trained medical doctor to diagnose the complication and determine an appropriate treatment. Hence, a problem that arises in a hospital-type environment is that the caregiver personnel such as nurses and other individuals who attend or visit a patient may not recognize, or even look for, or be aware of, observable signs that suggest the presence of complications. Such signs may easily be overlooked.

Accordingly, it is desirable to alert virtually all attending personnel in a caregiver medical facility and, where feasible, casual visitors and patients too, to look for, and to be aware of, and to note signs or symptoms or changes in signs of complications which are associated with particular diagnosed or suspected medical problems. Noticing identified, relevant signs would enable attending caregiver personnel to more quickly recognize a possible or foreseeable problem. Such recognition makes it possible to promptly alert the responsible, attending physician to possible serious complications. This could implement earlier detection of complications which were initially undetected or undetectable when the initial diagnosis was made.

In a typical hospital environment, a patient's attending physician may examine a patient during “rounds,” such as a morning or evening “round.” Thus, there can be a significant delay in recognizing and acting on a patient's complications. Hence, providing a system by which the usual caregiver attendants or visitors are made aware of arising complications and can give a suitable alarm when appropriate might avoid or minimize patient injuries.

An example of a situation in which an initial diagnosis is made and a subsequent developing complication is not detected early enough to reduce a resulting injury by quicker treatment, may be present in the diagnosis of meningitis. In some forms of meningitis, there is a risk of developing a cerebral edema. A developing edema may not be apparent in the initial diagnosis and the initial treatment of the meningitis. There are some classical signs suggesting the onset of cerebral edema, such as an irregular heartbeat, abnormal respiratory patterns, and an abnormal brain image observable in a CT scan, or MRI or ultrasound test. Separately, these signs may be disregarded or their significance may not be understood or may be explained away by other factors. The combination is significant. Thus, these signs, when considered separately, by separate people, may not alert them to a complication. But, when put together in one place, and considered together by one knowledgeable person, the combination may indicate that there might be a developing cerebral edema which should be treated soon to reduce or avoid brain damage. Waiting until the next routine “round” by the attending physician may be too long to take satisfactory preventative action.

As is known, brain edema increases the pressure within the skull. Thus, when the intracranial pressure exceeds the patient's blood pressure, blood and sufficient oxygen may not get to the neurons. That may result in death thereof. Consequently, early recognition and implementation of treatments for cerebral edema is essential to minimize or prevent damage to the brain.

Cerebral edema may be indicated on a CT scan or MRI by tissue herniating into a space in the skull where such tissue does not belong. A CT scan may indicate crowding in the base of the brain. The pressure begins to push the brain stem into the base of the skull so that vital functions regulated by the brain, such as heart rate and respiratory rate, can become disordered. These disorders can be signs of incipient cerebral edema.

The disturbance of the breathing pattern of the patient, such as respiratory rate cycles of rapid and slow breathing, may be observable to a visitor, e.g., an attendant visiting the patient for some purpose. Similarly, an irregular heart rate, known as a cardiac arrhythmia, may be an observable sign to another attendant routinely taking a patient's pulse or blood pressure. An MRI result may not be recognized as a sign by another technician. Three different people may each have observed only one of these signs. But, none of the three may recognize the significance of the particular signs which that person observed.

Listed together, and called to the attention of all observers, as possible signs of a developing cerebral edema because of the meningitis, the three signs of cerebral edema, namely, an irregular heartbeat, an abnormal respiratory pattern, and an abnormal MRI could be a warning. When these signs are viewed and considered separately by different people, they may not alert any of those people to the presence of the serious complication. But, when viewed together, in one easily seen place, they may raise an alarm to busy caregiver personnel such as nurses, nurses' aides, etc., who visit the patient. Otherwise, the personnel may not look for, or note or pay attention to the presence of these relevant signs. Hence, it is desirable to constantly call the attention of substantially all such personnel, when they attend to a patient, to look for and to be aware of specific, significant signs.

Routine comparisons of observed signs with a prioritized list of signs of complications that are associated with a particular patient's problem could cause the casual observer, even an untrained observer, to look for the signs and to note the signs. Thus, a warning can be provided to a responsible physician when the signs are noted. The significance of an observed sign appearing on a list can signal the viewer to pay attention, and to give immediate consideration to a possible complication. That early warning can implement early treatments that may be needed.

The foregoing is only an example of a situation where visitor observations of a patient, on a routine or random basis, which note and collect observations of signs or symptoms, can enable a suitably trained attending caregiver as well as an attending physician, to recognize complications and promptly take steps to instigate treatments. These noted observations by nurses and other visitors would also avoid waiting for regular, e.g. once- or twice-a-day routine examinations by an attending physician in a typical hospital to detect complications, and minimize injuries caused by, a serious complication.

The present invention provides for each patient a quickly observable, short or concise list of at least some of the more important signs or symptoms of complication risks which might be associated with, or which develop in response to, that particular patient's diagnosed or suspected maladies. The list may be constantly, routinely checked against the patient's observed conditions by all, or by many, of the caregiver personnel who visit a patient during a hospital stay. That could include nurses, nurses' aides, various technicians, helpers or service people, physicians and even patients, who normally would be unaware of the complication risks and the significance of observable signs.

By utilizing a concise list, preferably on a single sheet, where possible, for noting complication signs and changes in signs, a quick glance at the sheet may be all that is needed to understand and consider the patient's situation. Further, collecting a number of such sheets, from many patients, can support a study of common, observable signs which suggest particular complications that are associated with, or result from particular maladies. Hence, a single system of noting and recognizing significant or “key” signs that commonly are associated with specific medical problems can greatly assist a medical institution and its staff to more quickly recognize and deal with injury-causing complications.

### SUMMARY OF THE INVENTION

The invention herein contemplates a method by which actual or developing complications of pre-diagnosed or suggested medical problems may be more rapidly detected in a hospital-type environment by providing a chart with a concise list of complications that are associated with, or which may develop from, a particular diagnosed malady together with a list of significant signs or symptoms of the listed complications. That list is provided for particular patients and is made available at the sites of those patients so that the lists are called to the attention of visitors or attendants and consequently, observations of the listed signs may be noted. The listed complications and the listed “key” signs of them preferably are put in prioritized order.

Complication risks associated with an initially diagnosed problem can be based on the patient's physical condition and the patient's medical history, laboratory and other tests and the attending physician's experience, etc. Such risks are typically accompanied by observable signs or symptoms. Thus, it is contemplated that almost any of a hospital's personnel, such as nurses, nurse aides, technicians, and other visitors to a patient, with minimal training, will be reminded by the list on a chart to look for and note the presence of any of the listed signs and changes in those signs. Observations of any such signs can be noted on the charts. Thus, the charts can serve as an alarm or warning that can be used to alert an attending physician of the need to consider and diagnose the situation.

The accumulation or combination of the notations could indicate the possibility of the presence of, or developing, complications and indicate the need for more immediate review and diagnosis by a responsible, that is, trained attending physician. For example, referring to the above-mentioned cerebral edema complication, observation by a nurse or nurse's aide or by relatively untrained caregiver persons, of a patient's irregular breathing patterns and a irregular heart beat, and a technician's MRI test results, or other tests, which could be individually explained away, together might warn of a potential problem and indicate the need to obtain more immediate attention, rather than wait for a later routine examination. Also, that information would assist an attending physician to diagnose and prescribe a timely treatment.

The list of the risks of complications that are associated with a particular diagnosed malady can be provided on charts for each patient. In hospitals or other medical facilities, the charts could be kept at the site of the patient or kept in a computer database for display on computer monitors at the patient's site. Thus, the charts can be visible to, and routinely viewed, at a glance, by any caregiver person who is visiting or attending to the patient. This can be at the patient's room or bed or, on a computer monitor in a medical laboratory where tests are conducted or the results of tests observed.

An object of this invention is to enable earlier diagnoses of medical complications and problems which are related to, or are the results of, earlier diagnosed or suspected medical problems by providing lists of significant signs of complications that are associated with those particular problems for review and checking by persons visiting or attending the patient, as well as by the patient. Thus, signs or symptoms of possible complications can be quickly noted and considered, without waiting for more formal, but less frequent, examinations which otherwise would occur.

Another object of this invention is to enable caregiver personnel who are not particularly trained for diagnoses purposes, to be aware of, and to observe, even casually, and to note the presence of, and the changes in, signs of complications which may be associated with a particular diagnosed condition of the patient. Consequently, this enables almost all visitors, e.g. hospital personnel to more quickly call these significant signs to the attention of an appropriate physician for consideration of, and possible earlier treatment of injurious complications.

Yet another object of the present invention is to quickly put together in one place, e.g. on one sheet of paper, observations of signs of complications associated with diagnosed maladies or the worsening of the complications or maladies, so that the appropriate caretakers will be alerted to look for the combination of the signs and changes in the signs, and become aware of a possible medical problem that requires consideration and immediate treatment.

In general, the invention contemplates and has as its object, to collect information about significant, observable signs or symptoms in one easily viewable place, such as on one sheet of paper, or on one image of a computer monitor so that the collection of information can alert an appropriate caregiver to an existing or developing medical problem that requires attention.

These and other objects and advantages of this invention will become apparent upon reading the following description of which the attached drawing forms a part.

### DESCRIPTION OF THE DRAWINGS

FIG. 1 schematically illustrates a sample chart upon which signs of a cerebral edema complication, which is associated with meningitis, are presented for routine checking by persons that happen to visit or observe a particular meningitis patient in, for example, a hospital setting.

FIG. 2 schematically illustrates a sample chart presenting signs of an abdominal surgery complication that might occur as a result of such surgery.

FIG. 3 schematically illustrates an example of a chart in which the signs relating to unnamed complications to a particular, unnamed medical problem are presented.

FIG. 4 schematically illustrates a sample chart for monitoring changes in symptoms or signs for a heart attack patient. The chart presents the patient's significant base (i.e. normal) before the heart attack signs and abnormal signs detected at time of admission to the hospital for comparisons with later observed signs that are changed and therefore might indicate developing complications or worsening conditions.

### DETAILED DESCRIPTION OF THE PREFERRED EMBODIMENTS

Various medical illnesses and injuries are frequently accompanied by, or associated with, possible complications which may not be immediately diagnosed. Some developing complications are not observable or are otherwise not observed when an initial diagnosis of the patient's problem is made. In such instances, the presence or development of associated complications may not be detected soon enough to provide treatments which might have prevented or minimized the effects of an associated complication.

In many hospital environments, a patient may be visited infrequently, such as once- or twice-a-day, by a knowledgeable, attending physician. Sometimes the patient may simply be allowed to rest for some long periods of time in order to recover from a particular medical problem, without being re-diagnosed for complications.

When a patient, who is suffering from a diagnosed or suspected malady, develops a serious complication that is clearly observable, attention can be given to the patient. However, where the complication is latent or not quickly or readily diagnosed, delays in recognizing the complication or the possibility of the complication occurring in that particular patient because of that particular diagnosed malady, the delay may result in harm to the patient which might have been avoided by earlier recognition and treatment.

In some instances, signs of an impending or developing complication may be observed but are either disregarded or explained away. That is, it may take a collection or assembly of signs to give a warning of an oncoming complication. Yet, since a patient, in a hospital-type environment, may be viewed in the course of a day by a number of different caretaker persons, each viewer may observe a particular sign. But, no one compiles all the observed signs together into one combination which, together, might strongly indicate an oncoming serious complication problem.

The present invention contemplates providing a quickly viewable chart at a site close to the patient, as for example, a chart located in the patient's room or on the door of the room or upon the bed or at the adjacent nurse's station, or the like. As another example, the chart may be displayed on a computer monitor located physically near the patient or even remotely at a nurse's or laboratory site. If the chart is displayed on a computer monitor, a central monitor review site may also be provided so that a number of such charts may be periodically reviewed at the central site by a knowledgeable physician or person who can recognize the possible significance of changing conditions of a number of patients and, accordingly, be alerted to the need to take action or to alert their particular attending physicians.

The chart provides a concise list of possible complications that might occur for the particular patient's diagnosed or suspected malady. Consequently, any one of the hospital's personnel who happens to visit the patient and who observes the patient may glance at and mentally check the items on the list against the patient's condition. Then that person may note relevant observations on the chart. Hence, the list of the various signs or symptoms makes the visitor aware of the key signs which may signify a complication. Otherwise, the visitor might not look for, or notice, or even be aware of such signs.

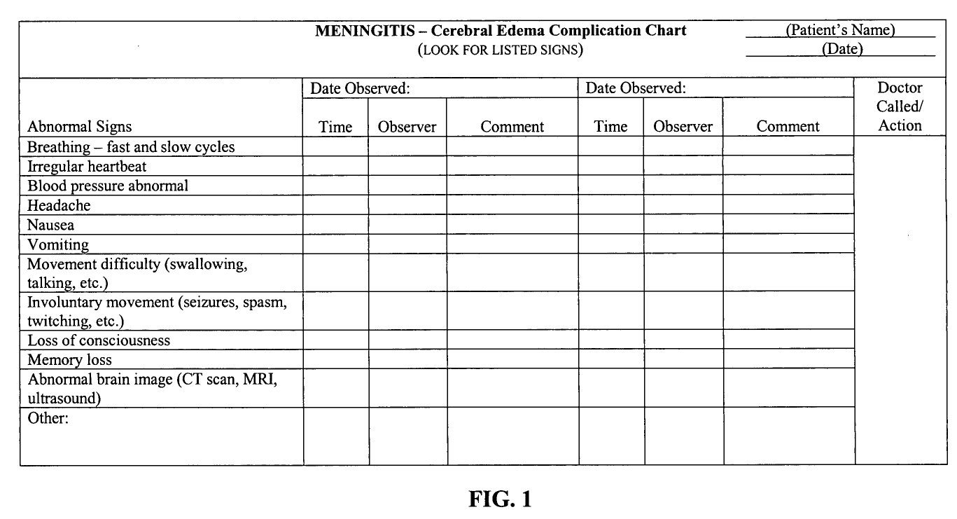

FIG. 1 schematically illustrates a sample chart which might be useable with a patient who has been diagnosed with a form of meningitis. As mentioned, certain forms of meningitis can result in a cerebral edema, that is, a swelling of the brain. Certain significant or “key” signs, when considered by a knowledgeable attending physician or similar medically trained caregiver, can suggest an oncoming cerebral edema. Hence, the chart contains, for illustration purposes, a number of common or “key” signs or symptoms of a developing cerebral edema. For example, the list includes an abnormal breathing pattern, an irregular heartbeat, and an abnormal MRI or CT or ultrasound scan, and other readily observable physical conditions. Further, significant signs also may be listed. Also, the date, time and observer may be listed along with comments or explanations of what was observed. In addition action taken, such as calling a doctor may be included to remind the observer about what to do with the observed information.

As illustrated, the chart may include the term “irregular heartbeat” which is detectable in the routine taking of a patient's pulse, and blood pressure from taking a routine blood pressure reading. Taking pulse and pressure readings are common events during a day in a hospital stay. Abnormal readings, together with other signs, may suggest a complication.

MRI tests, CT tests, ultrasound tests, X-rays, and other tests conducted with laboratory equipment are illustrated. Technicians or physicians conducting such tests and/or responsible nurses or other such responsible caregiver personnel, observing the results, may check the list and note abnormalities.

By way of example, an abnormal MRI would be indicated by a showing of tissue herniating into spaces not normally occupied by such tissue and/or the crowding of tissue in the base of the skull or tonsils of cerebellum at the tip of the spinal canal (referred to as an Arnold Chari condition). The technician observing the abnormality can note the findings on a computer displaying the patient's chart, or on the physical paper chart that may accompany the patient.

The chart may contain columns for time notations, and viewer's initials or name, with comments. Thus, changes in observed conditions noted at different times and days will help to evaluate the development or the worsening of a particular condition observed at a previous time.

The combination of the observations concisely noted or recorded in one place, e.g. preferably on one sheet or image of a chart, will, first, remind the visitor to look for the listed conditions and, second, to record the presence of those conditions when observed. Changes in the condition, e.g. worsening or improving or stable will then be apparent at a glance.

Different visitors, who separately observe different signs, jointly provide their observations into a compiled list. This compilation gives added significance to the observed signs. When considered together, the group of signs that were observed may give a strong warning of a particular complication which is associated with a patient's diagnosed malady. That, in turn, enables a responsible attending physician or a knowledgeable nurse, to immediately make a judgment concerning the complication and to determine the need for treatments and the type of treatments that should be given and, particularly, when treatments should begin. In many instances, the timing of a treatment needed for a developing or developed complication which had previously been undetected, may be critical to control the complication or to minimize injuries resulting therefrom.

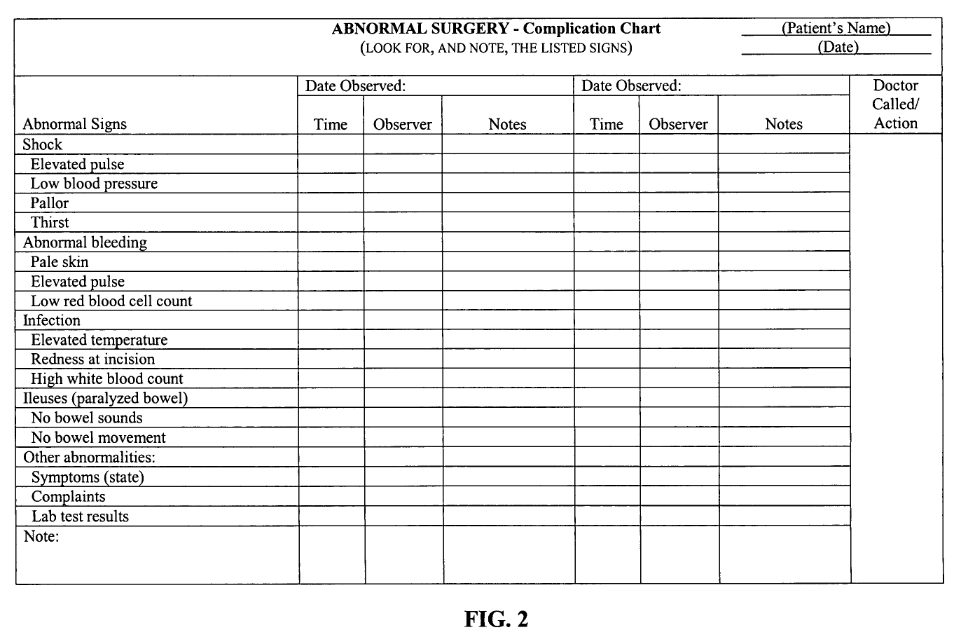

FIG. 2 schematically illustrates a second example of a complication chart which lists signs of several complications which are associated with, or result from, abdominal surgery. In this example, the chart indicates complication-related signs of shock, abdominal bleeding, infection, etc., and provides places to note other symptoms and complaints and laboratory test results that might be more specific to a particular patient.

The chart shown in FIG. 2, provides a list which alerts the medical or caretaker personnel to be aware of, and look for, such signs and to compare the list with the specific patient's condition whenever viewing that patient. It also provides a place for the viewer to mark the presence of observed signs and to comment upon whether the condition is changing. Notes may be added to indicate, for example, that some signs or the intensity of some signs may not be abnormal for that particular patient.

The accumulation of information may provide for indications of the possible causes of the abnormal finding or worsening abnormal finding which may lead to a different or further diagnosis of the patient's problem. Thus, the list may be “tailored” for each specific patient or be made to cover a number of patients.

The items on the chart should, where possible, list the more dangerous and more treatable conditions, particularly those conditions that would require earlier attention than other conditions. Essentially the chart is for providing an alarm or warning device rather than for providing a complete diagnostic tool.

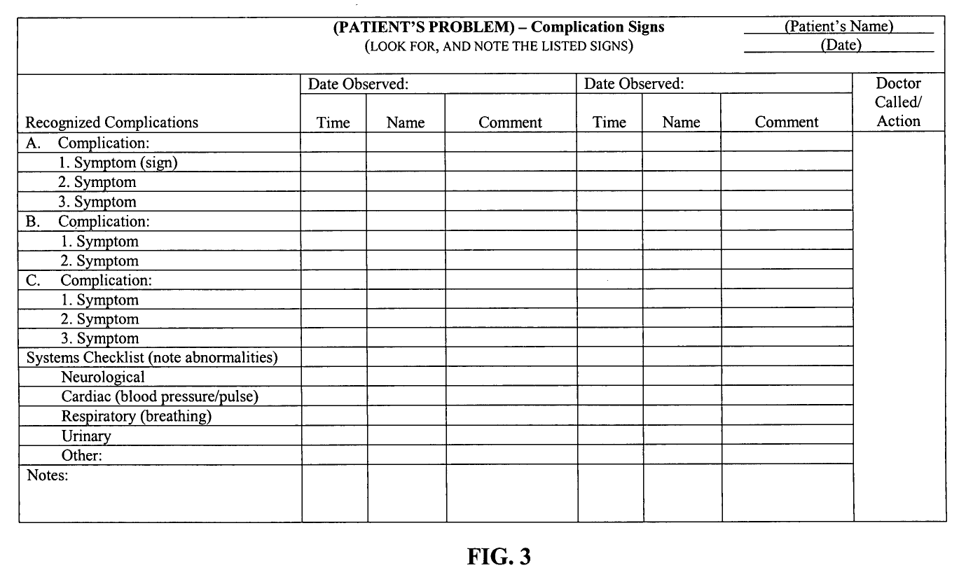

FIG. 3 illustrates a chart which schematically indicates a number of complications, which are not specifically identified on this example. The user of this would list the “key” symptoms or signs of the significant or likely complications. The chart also includes a “checklist” for reminding the observer of the various items to observe and to note abnormalities in those items. These can be useful in many instances to better identify or indicate a developing complication, which can be a worsening of another complication or condition. Thus, other signs may be also included on the chart as appropriate.

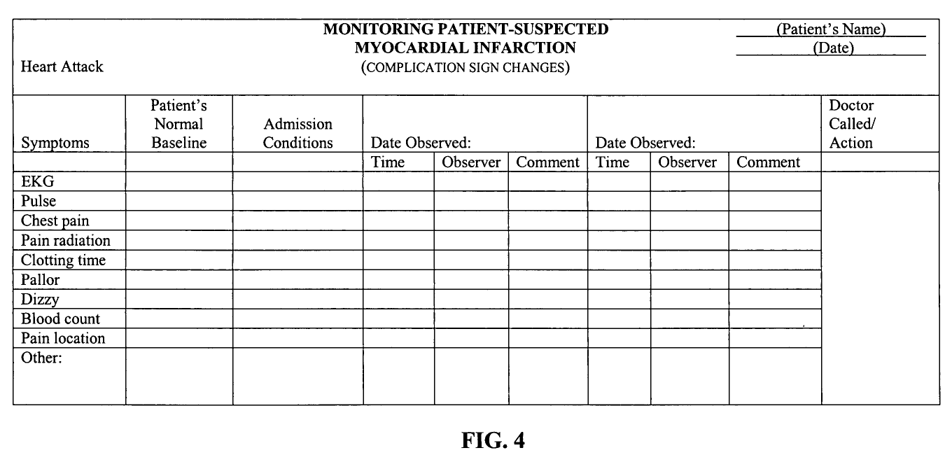

FIG. 4 illustrates schematically a sample chart for indicating a patient's “normal” or base signs, which might be statistically abnormal, but are actually normal for a specific person. For example, a particular patient might normally have an abnormally high blood pressure. Also, the chart may have abnormal signs that were detected at the initial admission examination and diagnosis of a suspected malady (e.g. heart attack) when the patent was admitted to the medical institution or doctor's care. Changes in those signs, whether base signs or initially considered signs, might help to evaluate the significance of later observed signs. For example, changes in relevant signs might warn of additional or newly developing complications or of serious worsening of the patient's condition. Thus, observing visitors seeing the notations of the already-known signs, would more likely pay attention to, and to note, observable changes.

The abnormal findings in the patient's condition may be noted on the charts by nurses or other caregiver personnel who would be empowered to make an appropriate notation for each finding. Significantly, the recognition of an abnormal finding would not necessarily depend on a physician's examination. Even the patient, under proper conditions, can be alerted to watch for listed conditions and signs and to make notes of them.

Once an observation of an abnormal condition or the worsening of the abnormality is noted on the chart, it then can be called to the attention of an appropriate caregiver physician. If an abnormal finding or listed sign is new or is worsening or is otherwise more significant in the context of finding other signs or abnormal conditions, an appropriate physician can be notified at once. That enables someone who has appropriate medical knowledge or experience, to determine the possible causes of the abnormal findings and to make an additional diagnosis or to prescribe an appropriate treatment.

The early recognition of the listed signs or abnormal findings helps determine whether the complication is sufficiently dangerous to require immediate attention, or at least to consider immediately for possible treatment or to change current treatings prescribed for an earlier diagnosis, etc. The combination of the significant, recognized signs or symptoms helps the attending physician to determine the likely cause of these signs. That enables an attending physician to be warned of and, therefore, to address, such causes and to take steps for prescribing treatments.

The illustrated charts described above are merely examples of listings and rankings of possible complications and signs of such complications that are associated with pre-diagnosed or suspected conditions of a patient. Those skilled in the art can make similar lists or charts which can be kept available for patients with pre-diagnosed or suspected conditions of various kinds. Even for conditions which are not yet diagnosed, such as where a patient is hospitalized for observation or an investigation is undertaken to determine the patient's particular problem, the charts may provide a list of observed symptoms which would be useful expediting consideration and treatment of the patient.

Preferably, the chart should be on a one-page sheet which concisely lists the important or “key” signs that are relevant to a particular patient's medical problem. That enables a quick scanning, at a glance, of the items to be aware of, and to be observed, by any person who attends to, or visits, the patient in the course of a day.

The charts are preferably made in advance by knowledgeable persons who are skilled in the pertinent medical arts. These would include physicians, medical writers, insurance organizations, pharmaceutical organizations, and the like, who are generally familiar with the kinds of complications, and the signs of such complications, which may be associated with various diagnosed maladies. In some cases, the observed abnormalities in the patients' condition, which are noted on the charts can assist in considering and in selecting treatments for a patient even when the particular malady is not yet determined.

Charts may be made on paper, for example, that are kept in a hospital or medical environment or clinic. Preferably, the chart is on one sheet. Alternatively, the charts may be kept in a computer having a monitor which is kept at a patient's location where a patient's chart can be read on the computer monitor. However, the chart can also be read on monitors placed at other locations such as an X-ray room or at a laboratory etc. When the chart is stored in a computer, a hard copy can be printed showing an appropriate complication and sign list that is relevant to a particular patient's maladies. Thus, depending upon the arrangement of a particular hospital or caregiver facility, a computer list can be accessed at a nurse's station near the patient or at other sites so that it can be accessed each time a nurse or other attendant visits the patient or a technician conducts tests or performs X-rays for a patient at another site to which a patient is moved or a test specimen taken for a laboratory test, etc. For example, when a technician visits a patient for the purpose of taking a blood sample or blood pressure or pulse, that particular person can refer to the chart located close to the patient or the patient's room, to mentally compare the listed signs with the observable condition of the patient and then to make notes on the chart to record the observations. Similarly, the results of a test can be entered at the laboratory or test equipment site.

The term caregiver, in general, as used herein refers to any person involved in providing medical type services. And the charts may be used with patients in hospitals, hospices, clinics, and other medical treatment facilities. The term complication is used in a general sense to refer to medical problems, such as those that relate to, or result from or are somehow associated with, other medical problems.

In some cases, a patient may have a condition which permits the patient to observe and compare the chart sign lists with his or her own physical condition. Therefore, the patient may make observations and notes thereon upon the chart. Then the chart, with the indications made by, among others, the patient, about the presence of signs and changes in the signs, can be observed at a glance by an appropriate caregiver person, such as a medical physician or nurse.

This invention may be further developed within the scope of the following claims. Having fully described an operative embodiment and a preferred embodiment of this invention, I now claim:

What is claimed is:

1. A method for alerting a caregiver to complications associated with a patient's medical problems comprising:
preliminarily diagnosing possible medical problems that the patient's condition indicates;
providing a list of complications which may be associated with or which may result from said diagnosed problems and including on the list possible observable signs which may indicate said complications, based upon the preliminary diagnosis;
providing such list to caregiver personnel at times when said personnel view and visit the patient, by making such list available to said personnel at the patient's site;
comparing the signs list with the observable condition of the patient, by individual caregiver personnel whenever observing the patient and noting the presence of, and changes in, observed signs that are on the list and alerting responsible caretakers to the appearance in the patient of listed signs and observed changes in signs;
diagnosing existing and developing complications and medical problems of the patient in response to the appearance of, or changes in, said listed symptoms.

2. A method as defined in claim 1, and routinely recording on the list any listed signs that are observed and any changes in such listed symptoms that are observed whenever a caregiver observes the patient,
whereby, a responsible caregiver may be alerted to an existing problem or an oncoming, developing problem.

3. A method as defined in claim 1, and including on said list a group of associated signs, prioritized in order of importance,
whereby, the group when observed will strongly suggest the presence of the complication.

4. A chart for warning of, and implementing the detection of, and the treatment of previously undiagnosed complications that may be associated with, or that result from, an earlier diagnosed medical problem of a hospitalized patient, comprising:
a list which identifies an earlier diagnosed problem or possible problem;
a list of complications which are associated with or which can result from that problem;
a list of observable signs associated with each listed complication and which may indicate the presence of, or the developing of, each of said complications;
with said chart normally, in use, being located at the site of the patient for routine review by any caretaker personnel, such as nurses and other hospital attendants, who may visit the patient's site from time to time, for comparing the listed signs with observed conditions of the patient;
whereby the presence of, and the changes in, the listed signs will enable a medical caretaker to alert an attending physician, to the presence of, or onset of, previously undiagnosed complications for an early diagnosis of, and consideration of treatment of such complications.

5. A chart as defined in claim 4, and wherein said list of signs includes observable abnormalities in the patient's physical condition as well as physical conditions known to present any of the listed complications.

6. A chart as defined in claim 5, and including the signs being organized in groups which groups of signs, when observed together, indicate the presence of a complication.

7. A chart as defined in claim 4, and including places for notations on the chart of the signs observed and any changes in the observed signs and when such observations occurred.

8. A chart as defined in claim 7, and including the list of said complications and the listing of signs of such complications being prioritized to emphasize complications which can injure a patient;
whereby observing the presence of such signs enables giving an appropriate alarm for immediate attention needed for the patient.

9. A chart for enabling medical caregiver attendants to provide an alarm to attending physicians and to warn attending physicians of a possible actual, or developing, previously undiagnosed complications associated with or resulting from a previously diagnosed or suspected medical problem suffered by a patient, comprising:
a substantially single sheet upon which the diagnosed or suspected medical problem is identified;
such sheet including a list of complications which are known to be associated with or to result from said medical problem; and a list of observable signs which are associated with the presence of, and the developing of each of the listed complications;
said chart being normally located at the site of the patient for routine observation and review by any patient attendant, such as a nurse, and other hospital persons and visitors, who visit the patient's site from time to time, and during such visit may routinely glance at, and compare the listed signs with the observed conditions of the patient;
such chart having a place for recordings of observed signs corresponding to the listed signs, and for changes in such signs observed during such visits;
whereby the presence of recorded signs and changes in recorded signs will alert attending hospital personnel to the possible occurrence of injurious complications and consequently serve to warn responsible attending physicians to detect such complications and consider treatments therefore, even before times of normal, routine examinations by the patient's physicians, as well as to enable the treating physicians to diagnose and prescribe timely treatments thereof.

10. A chart as defined in claim 9, and said sheet being in the form of a computer monitor screen image.

11. A method for providing a warning to an attending physician of the presence of, or developing of, a previously undiagnosed complication associated with or resulting from a previously diagnosed or suspected medical problem suffered by a hospitalized patient, comprising:
preparing a list of substantially one sheet in length, with a list of likely complications that may be associated with a diagnosed or suspected malady;
listing with said complications known signs in the patient's conditions which are associated with said complications;
positioning the chart at the location of the patient for viewing by visitors to the patient, including attending nurses and other hospital attendants and casual visitors, so that said visitors may view the list of signs on the chart and observe whether any such signs are apparent on the patient;
noting observations of any of the listed signs and changes therein on the chart so that the collection of notations of observed signs can be seen at a glance by a sufficiently medically knowledgeable visitor, such as a nurse and a treating physician, to recognize the significance of the signs and consequently to warn a responsible, treating physician to the possible occurrence of a complication and for the need of a timely consideration of treatments to minimize injury to the patient resulting from the complication.

12. A method as defined in claim 11 and including routinely comparing the list of signs by nurses and other hospital attendants and personnel with the observed conditions of the patient and changes in the conditions observed on the patient during such visits to the patient between normal examinations by the patient's attending physicians for noting occurrences of the signs and thereby enabling the prompt alerting of an attending physician to consider and to prescribe treatments for the patient.

13. A method as defined in claim 11, and wherein said chart is made visually observable to hospital attendants and visitors to a patient for reminding the attendants and visitors of the signs in the patient's condition to look at, and wherein places are provided on the chart for notations of observations of such signs and changes in the signs, during visits between routine examination visits of the patient's treating physician;
whereby observations of the signs and changes therein, serve to warn of a possible undetected complication or developing complication, so that an attending physician may be alerted to immediately consider and possibly prescribe treatments for potentially injurious complications.
